(19) United States
(12) Patent Application Publication
(10) Pub. No.: US 2022/0289782 A1
(43) Pub. Date: Sep. 15, 2022

(54) A Compound with High Anticoagulant Activity and Its Preparation Method and Application

(71) Applicant: SUZHOU WISMED PHARMACEUTICALS CO LTD, Suzhou (CN)

(72) Inventor: Peng Wang, Suzhou (CN)

(21) Appl. No.: 17/614,470

(22) PCT Filed: Jun. 24, 2020
(86) PCT No.: PCT/CN2020/098112

§ 371 Date: May 24, 2022

(30) Foreign Application Priority Data
May 28, 2019 (CN) 2019 10449774.5
Jun. 26, 2019 (CN) 2019 10561636.6

(51) Int. Cl. C07H 11/00

(52) U.S. Cl. CPC C07H 11/00

(57) ABSTRACT

The present invention relates to a high anticoagulation activity compound, a preparation method and an application, said compound being formed from a monosaccharide unit D, a monosaccharide unit E, a 6-position carbon oxygen-substituted monosaccharide unit F, a monosaccharide unit G and a monosaccharide unit H sequentially connected by glycosidic bonds, the bond three-dimensional configuration being a-D-glucose-(1→4)-0-p-D-glucuronic acid-(1→4)-0-a-D-(6-carbon oxygen-substituted)glucose-(1→4)-0-a-L-iduronic acid-(1-4)-0-a-D-(6-carbon oxygen-substituted)methyl glucose. The monosaccharide unit D is a glucose 2,6-O-sulfated group, the monosaccharide unit E is a glucuronidated group, the monosaccharide unit F is a glucose 2,3-0-6-carbon oxygen-substituted-sulfated group, the monosaccharide unit G is an L-iduronic acid 2-O-sulfated group, and the monosaccharide unit H is a glucose 2,3-0-6-carbon oxygen-substituted-sulfated group. The synthesized compound has higher anticoagulation activity than when the 6-positions of the F unit and H unit are oxygen.

### TECHNICAL FIELD

This invention belongs to the pharmaceutical field and relates to a new compound with high anticoagulant activity (similar to pentose). Specifically, it relates to a compound with high anticoagulant activity (similar to pentose), its preparation method and application.

### TECHNICAL BACKGROUND

Heparin is a kind of mucopolysaccharide sulfate extracted from animals, which has anticoagulant and antithrombotic effects. Due to the heparin-induced hemorrhage at the initial stage of its use, low-molecular-weight heparin (LMWH) appeared in the late 1980s, which can effectively reduce the risk of hemorrhage. However, as the raw material of LMWH is ordinary heparin, it is subject to the possibility of viral contamination of species at its source, which may cause some problems when using heparin or LMWH due to endogenous viruses. Meanwhile, due to biochemical pollution in the process of animal breeding and heparin extraction, the application of heparin is restricted to various degrees.

Over forty years ago, some European and American scientists, led by Lindahl and Choay, established that the effective fragment of heparin is a pentasaccharide structure containing iduronic acid when they studied the anticoagulant and antithrombotic effects of heparin. Since then, there has been an international upsurge in the synthesis of glycans with similar structures to heparin. Some scientists, led by Sinay and van Boeckel, have completed the complete chemical synthesis of pentose. European Patent EP0084999 and American Patent U.S. Pat. No. 4,818,816 have put forward the synthesis and pharmaceutical application of Fondaparinux sodium.

The Patent EP0165134 also describes the synthesis of oligosaccharide with antithrombotic activity: the compound composed of uronic acid and glucosamine with O-sulfate radical group introduced at the 3-position of glucosamine unit has strong anticoagulant activity. The Patents EP0301618 and EP0529175 describe sulfated glycosaminoglycan derivatives in which N-sulfate radical group, N-acetyl group or hydroxyl functional group have been replaced by alkoxy group, aryloxy group, arylalkoxy group or O-sulfate radical group. These compounds have beneficial antithrombotic properties, but when hydroxyl groups are completely replaced by alkoxy groups, aryloxy groups, arylalkoxy groups, etc., the side effects such as cardiovascular fibrosis will increase accordingly, and relevant reports have been published recently. The Patent (Application No. CN201310090934) discloses that a pentose compound based on fondaparinux sodium whose nitrogen-atoms are replaced by oxygen atoms has higher anticoagulant activity than fondaparinux sodium, and its synthesis is simplified, but there is still room for improvement for its anticoagulant activity.

### CONTENTS OF THE INVENTION

In order to overcome the defects in the existing technology, the present invention aims to provide a new anticoagulant compound with high activity, which is similar in structure to pentose, but the oxygen-atoms connected with the monosaccharide units F and H at the six-carbon position are converted into carbon-atoms to increase its anticoagulant activity.

For the above purpose, the technical solution adopted in this invention is: A compound with high anticoagulant activity is invented. It is a pentose compound and is formed by connecting monosaccharide units D, E, F, G and H in sequence through glycosidic bonds, and its spatial configuration of bond connections is α-D-glucose-(1→4)-O-β-D-glucuronic acid-(1→4)-O-α-D-(6-Carbon substitution for oxygen) glucose-(1→4)-O-α-L-iduronic acid-(1→4)-O-α-D-(6-Carbon substitution for oxygen) methylglucose; the said monosaccharide unit D is a glucose 2,6-O-sulfated group, the said monosaccharide unit E is a glucuronidated group, the said monosaccharide unit F is a glucose 2,3-O-6-Carbon substitution for oxygen-sulfated group, the said monosaccharide unit G is a L-iduronic acid 2-O-sulfated group, and the said monosaccharide unit H is a glucose 2,3-O-6-Carbon substitution for oxygen-sulfated group.

Optimally, it is an ionic compound and its anionic structural general formula is shown in formula (1):

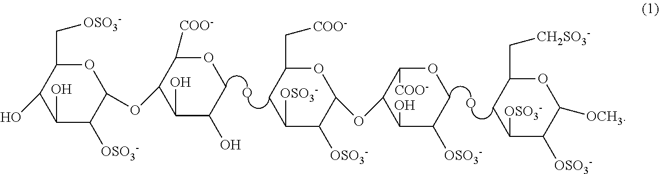

Further, its cations are selected from one or several kinds of cations such as potassium ion, sodium ion and hydrogen ion.

Another purpose of the present invention is to provide a method for synthesizing the said compound with high anticoagulant activity, and the said method is characterized in that a trisaccharide composed of monosaccharide units D, E and F is connected with a disaccharide composed of monosaccharide units G and H; or a disaccharide composed of monosaccharide units D and E is connected with a trisaccharide composed of monosaccharide units F, G and H; or a tetrasaccharide composed of monosaccharide units D, E, F and G is connected with a monosaccharide composed of monosaccharide unit H; or a disaccharide composed of monosaccharide units D and E is connected with a disaccharide composed of monosaccharide units F and G and then connected with a monosaccharide composed of monosaccharide unit H.

Optimally, it comprises the following steps:

A trisaccharide composed of monosaccharide units D, E and F is synthesized, and its structural general formula is shown in formula (2).

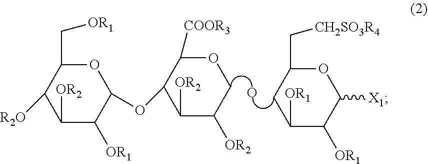

In formula (2), X₁ is selected from thioalkyl, thioaryl, halogen, trichloroiminoacetyl, phosphate ester, phosphate ester leaving group or n-pentenyl group, and its spatial configuration can be a or β; R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R₄ is alkyl;

A disaccharide composed of monosaccharide units G and H is synthesized, and its structural general formula is shown in formula (3).

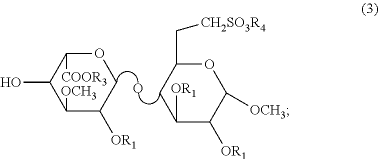

In formula (3), R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R4 is alkyl;

And then the said trisaccharide is connected to the said disaccharide.

Optimally, it comprises the following steps:

A tetrasaccharide composed of monosaccharide units E, F, G and H is synthesized, and its structural general formula is shown in formula (4).

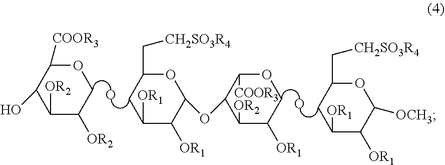

In formula (4), R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R4 is alkyl;

And then the said tetrasaccharide is connected to a monosaccharide;

The general structural formula of the said monosaccharide is shown in formula (5).

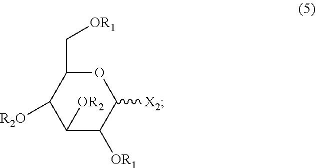

In formula (5), X₂ is selected from thioalkyl, thioaryl, halogen, trichloroiminoacetyl or n-pentenyl, and its spatial configuration is a or β; R₁ is selected from benzyl or substituted benzyl; R₂ is selected from alkyl acyl, aryl acyl or alkyl aryl acyl, or substituted alkyl acyl, aryl acyl or alkyl aryl acyl.

Optimally, the said monosaccharide unit D is derived from monosaccharides with the following structural general formula:

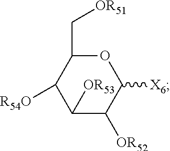

In the formula, X₆ is selected from thioalkyl, thioaryl, halogen, trichloroiminoacetyl or n-pentenyl, and its spatial configuration is a or β; R₅₁ is selected from benzyl or substituted benzyl; R₅₂ is selected from benzyl or substituted benzyl; R₅₃ is selected from alkyl acyl, aryl acyl, alkyl aryl acyl, allyl, allyl ether or p-Methoxybenzyl (PMB) protective group, or substituted alkyl acyl, aryl acyl or alkyl aryl acyl; R₅₄ is selected from alkyl acyl, aryl acyl or alkyl aryl acyl, or substituted alkyl acyl, aryl acyl or alkyl aryl acyl; R₅₁ and R₅₄ can form cyclic acetals or ketal.

Optimally, the said monosaccharide unit E is derived from monosaccharides with the following structural general formula:

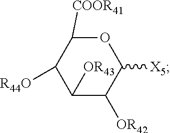

In the formula, X₅ is selected from thioalkyl, thioaryl, halogen, trichloroiminoacetyl or n-pentenyl, and its spatial configuration is a or β; R₄₁ is selected from benzyl or substituted benzyl; R₄₂ is selected from alkyl acyl, aryl acyl or alkyl aryl acyl, or substituted alkyl acyl, aryl acyl or alkyl aryl acyl; R₄₃ is selected from alkyl acyl, aryl acyl or alkyl aryl acyl, or substituted alkyl acyl, aryl acyl or alkyl aryl acyl; R₄₄ is selected from hydrogen, chloroacetyl or acetyl propionyl.

Optimally, the said monosaccharide unit F is derived from monosaccharides with the following structural general formula:

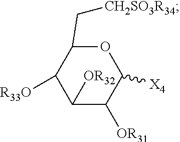

In the formula, X₄ is selected from p-methoxyphenyl or p-methoxybenzyl, and its spatial configuration is a or β; R₃₁ is selected from benzyl or substituted benzyl; R₃₂ is selected from benzyl or substituted benzyl; R₃₃ is selected from hydrogen or acetyl propionyl; R34 is alkyl.

Optimally, the said monosaccharide unit G is derived from monosaccharides with the following structural general formula:

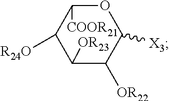

In the formula, X₃ is selected from thioalkyl, thioaryl, trichloroiminoacetyl or n-pentenyl, and its spatial configuration is a or β; R₂₁ is selected from benzyl or substituted benzyl; R₂₂ is selected from benzyl or substituted benzyl, alkyl acyl, aryl acyl or alkyl aryl acyl or substituted alkyl acyl, aryl acyl or alkyl aryl acyl; R₂₃ is selected from substituted benzyl; R₂₄ is selected from hydrogen or acetyl propionyl.

Optimally, the monosaccharide unit H is derived from monosaccharides with the following structural general formula:

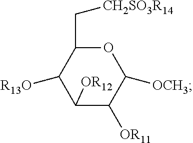

In the formula, R₁₁ is selected from benzyl or substituted benzyl; R₁₂ is selected from benzyl or substituted benzyl; R₁₃ is selected from hydrogen or acetyl propionyl; R₁₄ is alkyl

Another purpose of the present invention is to provide an application of the said compound with high anticoagulant activity to serve as an active ingredient in drugs for blood coagulation dysfunction.

Optimally, the said compound with high anticoagulant activity is mixed with at least one pharmaceutical molding agent in a unit dose of 0.1-10 mg.

Because of the application of the said technical solution, the present invention has the following advantages compared with the existing technology: the invented compound with high anticoagulant activity is basically consistent with the minimum sequence unit structure of heparin. N-sulfate radical and N-acetyl groups are replaced by O-sulfate radical groups, and the O-sulfate radical group is introduced to the 3-position of the end-position glucose unit, so that the synthesized compound has high anticoagulant activity.

### BRIEF DESCRIPTION OF THE FIGURES

FIG. 1 shows the synthesis process of building block H in Example 1;

FIG. 2 shows the synthesis process of building block G in Example 2;

FIG. 3 shows the synthesis process of building block F in Example 3;

FIG. 4 shows the synthesis process of building block D in Example 4;

FIG. 5 shows the synthesis process of building block E in Example 5;

FIG. 6 shows the synthesis process of building block GH in Example 6;

FIG. 7 shows the synthesis process of building block EF in Example 7;

FIG. 8 shows the synthesis process of building block DEF in Example 8;

FIG. 9 shows the synthesis process of fully-protected pentose in Example 9;

FIG. 10 shows the synthesis process of API with fully-protected pentose in Example 10;

FIG. 11 shows the synthesis process of substitutable building block E′ in Example 11;

FIG. 12 shows the synthesis process of disaccharide E′F with substitutable building block E5′ and building block F5 in Example 12;

FIG. 13 shows the synthesis process of tetrasaccharide EFGH with building block E′F3 and disaccharide building block GH in Example 13;

FIG. 14 shows the synthesis process of pentose DEFGH with building block EFGH and monosaccharide building block D8 in Example 14.

### DETAILED MODE OF EXECUTION

A compound with high anticoagulant activity is invented. It is a pentose compound (a pentose compound derivative indeed) and is formed by connecting monosaccharide units D, E, F, G and H in sequence through glycosidic bonds, and it is characterized in that its spatial configuration of bond connections is α-D-glucose-(1→4)-O-β-D-glucuronic acid-(1→4)-O-α-D-(6-Carbon substitution for oxygen) glucose-(1→4)-O-α-L-iduronic acid-(1→4)-O-α-D-(6-Carbon substitution for oxygen) methylglucose; the said monosaccharide unit D is a glucose 2,6-O-sulfated group, the said monosaccharide unit E is a glucuronidated group, the said monosaccharide unit F is a glucose 2,3-O-6-Carbon substitution for oxygen-sulfated group, the said monosaccharide unit G is a L-iduronic acid 2-O-sulfated group, and the said monosaccharide unit H is a glucose 2,3-O-6-Carbon substitution for oxygen-sulfated group. In this way, the compound is basically consistent with the minimum sequence unit structure of heparin. N-sulfate radical and N-acetyl groups are replaced by O-sulfate radical groups, and the O-sulfate radical group is introduced to the 3-position of the end-position glucose unit, so that the synthesized compound (similar to pentose) has high anticoagulant activity.

The said compound with high anticoagulant activity is an ionic compound, and its anionic structural general formula is shown in formula (1):

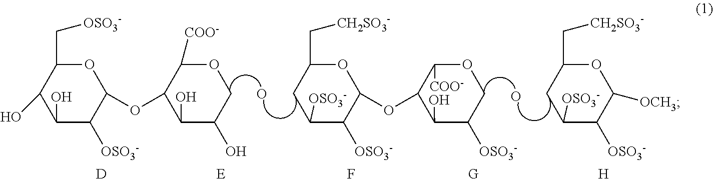

Its cations are selected from one or several kinds of cations such as potassium ion, sodium ion and hydrogen ion; the structure can be combined with ATIII to form a Compound-ATIII complex, and then combined with Xa factor to achieve the purpose of anticoagulation, so it has higher activity compared with the previous anticoagulant pentose.

The synthetic method of the said compound with high anticoagulant activity is characterized in that a trisaccharide composed of monosaccharide units D, E and F is connected with a disaccharide composed of monosaccharide units G and H; or a disaccharide composed of monosaccharide units D and E is connected with a trisaccharide composed of monosaccharide units F, G and H; or a tetrasaccharide composed of monosaccharide units D, E, F and G is connected with a monosaccharide composed of monosaccharide unit H; or a disaccharide composed of monosaccharide units D and E is connected with a disaccharide composed of monosaccharide units F and G and then connected with a monosaccharide composed of monosaccharide unit H.

Specifically, the optional methods are as follows:

(1) A trisaccharide composed of monosaccharide units D, E and F is synthesized, and its structural general formula is shown in formula (2).

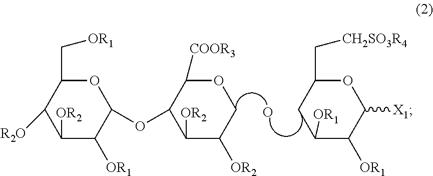

In formula (2), X₁ is selected from thioalkyl, thioaryl, halogen, trichloroiminoacetyl, phosphate ester, phosphate ester leaving group or n-pentenyl group, and its spatial configuration can be a or β; R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R₄ is from methyl, ethyl or other conventional alkyl groups.

A disaccharide composed of monosaccharide unit G and 6-Carbon substitution for oxygen-monosaccharide unit H is synthesized, and its structural general formula is shown in formula (3).

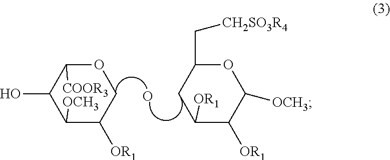

In formula (3), R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R₄ is selected from methyl, ethyl or other conventional alkyl groups.

And then the said trisaccharide is connected to the said disaccharide.

(2) A tetrasaccharide composed of monosaccharide unit E, 6-Carbon substitution for oxygen-monosaccharide unit F, monosaccharide unit G and 6-Carbon substitution for oxygen-monosaccharide unit H is synthesized, and its structural general formula is shown in formula (4).

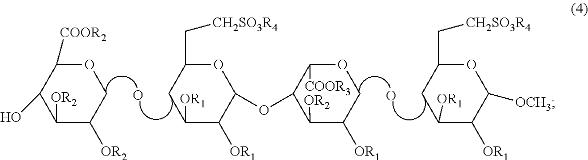

In formula (4), R₁ is selected from benzyl or substituted benzyl; R₂ is selected from alkyl acyl, aryl acyl or alkyl aryl acyl, or substituted alkyl acyl, aryl acyl or alkyl aryl acyl; R₃ is selected from benzyl or substituted benzyl; R₄ is selected from methyl, ethyl or other conventional alkyl groups.

And then the said tetrasaccharide is connected to a monosaccharide;

The general structural formula of the said monosaccharide is shown in formula (5).

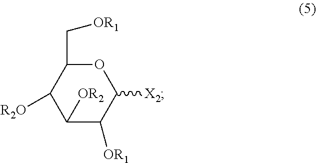

In formula (5), X₂ is selected from thioalkyl, thioaryl, halogen, trichloroiminoacetyl or n-pentenyl, and its spatial configuration is a or β; R₁ is selected from benzyl or substituted benzyl; R₂ is selected from alkyl acyl, aryl acyl or alkyl aryl acyl, or substituted alkyl acyl, aryl acyl or alkyl aryl acyl.

It can also be adjusted according to actual raw materials and processes (to obtain different intermediates), and can be constructed according to the said monosaccharide units, specifically as follows:

(1) The monosaccharide unit D is derived from the monosaccharide with the following structural general formula (defined as building block D), that is, the corresponding monosaccharide unit D can be obtained by reacting the monosaccharide with the following structural general formula with other monosaccharides, the same below:

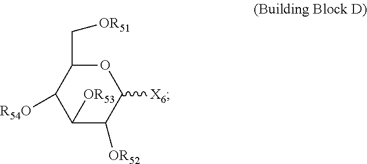

In the formula, X₆ is selected from thioalkyl, thioaryl, halogen, trichloroiminoacetyl or n-pentenyl, and its spatial configuration is a or β; R₅₁ is selected from benzyl or substituted benzyl; R₅₂ is selected from benzyl or substituted benzyl; R₅₃ is selected from alkyl acyl, aryl acyl, alkyl aryl acyl, allyl, allyl ether or p-Methoxybenzyl (PMB) protective group, or substituted alkyl acyl, aryl acyl or alkyl aryl acyl; R₅₄ is selected from alkyl acyl, aryl acyl or alkyl aryl acyl, or substituted alkyl acyl, aryl acyl or alkyl aryl acyl; R₅₃ and R₅₄ can be the same or different; R₅₁ and R₅₄ can form cyclic acetals or ketal.

(2) The said monosaccharide unit E is derived from monosaccharides with the following structural general formula:

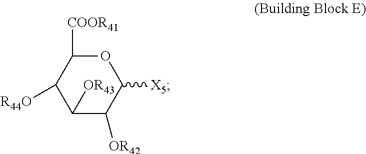

In the formula, X₅ is selected from thioalkyl, thioaryl, halogen, trichloroiminoacetyl or n-pentenyl, and its spatial configuration is a or β; R₄₁ is selected from benzyl or substituted benzyl; R₄₂ is selected from alkyl acyl, aryl acyl or alkyl aryl acyl, or substituted alkyl acyl, aryl acyl or alkyl aryl acyl; R₄₃ is selected from alkyl acyl, aryl acyl or alkyl aryl acyl, or substituted alkyl acyl, aryl acyl or alkyl aryl acyl; R₄₄ is selected from hydrogen, chloroacetyl or acetyl propionyl.

(3) The said 6-Carbon substitution for oxygen-monosaccharide unit F is derived from monosaccharides with the following structural general formula:

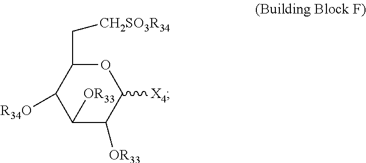

In the formula, X₄ is selected from p-methoxyphenyl or p-methoxybenzyl, and its spatial configuration is a or β; R₃₁ is selected from benzyl or substituted benzyl; R₃₂ is selected from benzyl or substituted benzyl; R₃₃ is selected from hydrogen or acetyl propionyl; R₃₁ and R₃₄ can form cyclic acetals or ketal.

(4) The said monosaccharide unit G is derived from monosaccharides with the following structural general formula:

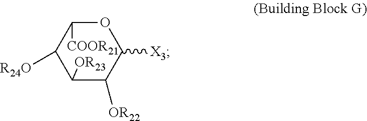

In the formula, X₃ is selected from thioalkyl, thioaryl, trichloroiminoacetyl or n-pentenyl, and its spatial configuration is a or β; R₂₁ is selected from benzyl or substituted benzyl; R₂₂ is selected from benzyl or substituted benzyl, alkyl acyl, aryl acyl or alkyl aryl acyl or substituted alkyl acyl, aryl acyl or alkyl aryl acyl; R₂₃ is selected from methoxy; R₂₄ is selected from hydrogen or acetyl propionyl.

(5) The said 6-Carbon substitution for oxygen-monosaccharide unit H is derived from monosaccharides with the following structural general formula:

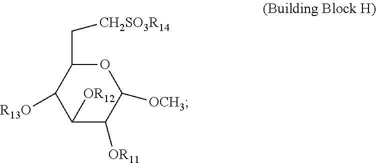

In the formula, R₁₁ is selected from benzyl or substituted benzyl; R₁₂ is selected from benzyl or substituted benzyl; R₁₃ is selected from hydrogen or acetyl propionyl; R₁₄ is selected from methyl, ethyl or other alkyl, and R₁₁ and R₁₂ can be the same or different.

(6) Synthesize or use a disaccharide intermediate with the following structural general formula:

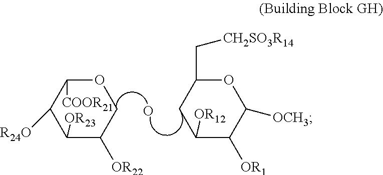

In the formula, R₁₁, R₁₂ and R₁₄ are as defined in building block H; R₂₁, R₂₂, R₂₃ and R₂₄ are as defined in building block G.

(7) Synthesize or use a disaccharide intermediate with the following structural general formula:

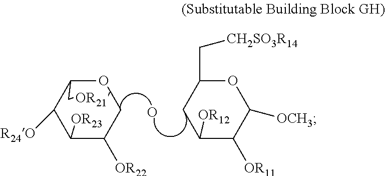

In the formula, R₁₁, R₁₂ and R₁₄ are as defined in building block H; R₂₂ and R₂₃ are as defined in building block G, and R_{21′} and R_{24′} are selected from alkyl, aryl or substituted aryl acetals or ketal.

(8) Synthesize or use a disaccharide intermediate with the following structural general formula:

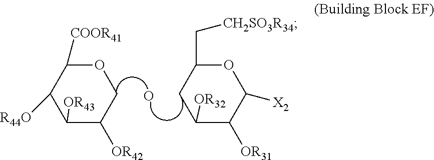

In the formula, X₂ is selected from p-methoxyphenyl and p-methoxybenzyl, which are a or β-type connection; R₃₁, R₃₂ and R₃₄ are as defined in building block F, and R₄₁, R₄₂, R₄₃ and R₄₄ are as defined in building block E.

(9) Synthesize or use a disaccharide intermediate with the following structural general formula:

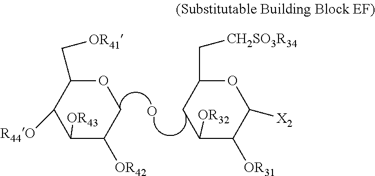

In the formula, X₂ is selected from alkoxy, or aryloxy and substituted aryloxy, which are a or β-type connection; R₃₁, R₃₂ and R₃₄ are as defined in building block F, R₄₂ and R₄₃ are as defined in building block E, and R_{41′} and R_{44′} are selected from alkyl, aryl or substituted aryl acetals or ketal.

(10) Synthesize or use a trisaccharide intermediate with the following structural general formula:

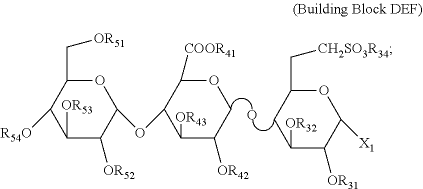

In the formula, X₁ is selected from thioalkyl, thioaryl, halogen, trichloroiminoacetyl, phosphate ester, related phosphate ester leaving group or n-pentenyl, and its spatial configuration is a or β;

R₃₁, R₃₂ and R₃₄ are as defined in building block F, R₄₁, R₄₂ and R₄₃ are as defined in building block E, and R₅₁, R₅₂, R₅₃ and R₅₄ are as defined in building block D.

(11) Synthesize or use a tetrose intermediate with the following structural general formula:

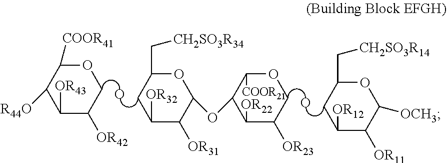

In the formula, R₁₁, R₁₂ and R₁₄ are as defined in building block H; R₂₁, R₂₂ and R₂₃ are as defined in building block G, R₃₁, R₃₂ and R₃₄ are as defined in building block F, and R₄₁, R₄₂, R₄₃ and R₄₄ are as defined in building block E.

(12) Synthesize or use a tetrose intermediate with the following structural general formula:

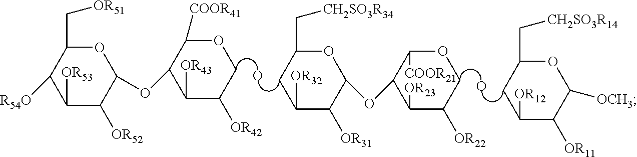

In the formula, X is selected from hydroxyl or alkoxy; R₁₁, R₁₂ and R₁₄ are as defined in building block H, R₂₁, R₂₂ and R₂₃ are as defined in building block G, R₃₁, R₃₂ and R₃₄ are as defined in building block F, R₄₁, R₄₂ and R₄₃ are as defined in building block E, and R₅₁, R₅₂, R₅₃ and R₅₄ are as defined in building block D.

The said pentose compound can also be formed by removing the corresponding protecting group from pentose with the following structure:

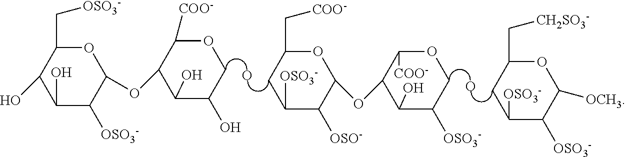

The said compound (similar to pentose) can be used as active ingredients in drugs related to blood coagulation dysfunction, that is, it can be used in the pharmaceutical composition related to blood coagulation dysfunction that contains salt forms of pharmaceutically acceptable bases as active ingredients or acid forms of the said compound, as well as medicinal nontoxic agents combined or mixed with it (that is, the active ingredients are mixed with at least one medicinal molding agent to form a pharmaceutical composition). In the composition, the dosage unit contains 0.1-10 mg of active ingredients (i.e., the said pentose compound is mixed with at least one pharmaceutical molding agent in a unit dosage of 0.1-10 mg).

The abbreviations used in this application are as follows: Ac: acetyl; Bn: benzyl; CAN: cerium (IV) ammonium nitrate; DMF: N,N-dimethylformamide; NIS: N-iodosuccinimide; TBAF: Tetrabutylammonium fluoride; TBSOTf: Tert-butyldimethylsilyl trifluoromethanesulfonate; TEMPO: 2,2,6,6-tetramethyl-1-piperidinyloxy; TFA: trifluoroacetic acid; TFAA: trifluoroacetic anhydride; Tf: trifluoromethanesulfonyl; TMS: trimethylsilyl; p-TsOH: p-toluenesulfonic acid.

The invention will be further explained with examples below.

### EXAMPLE 1

This example provides a method for synthesizing building block H with a-methylglucose, as shown in FIG. 1 and under the following conditions: a) 1. PhCH (OMe)₂, p-TsOH, DMF, 50° C.; 2. NaH, BnBr, DMF, two-step yield 78%; b) 80% acetic acid, 70° C., 85%; c) 1. Tf₂O, DMAP; 2. AC₂O, DMAP; 3. BuLi, CH₃SO₃Et, −20° C.; 4. CH₃ONa, CH₃OH; four-step yield 65%.

Preparation of H1: dissolve a-methylglucose (38.8 g) in DMF (400 ml), add p-toluenesulfonic acid monohydrate (4 g) and 8.9 ml benzylidene reagent PhCH (OMe)₂ at room, and then react under reduced pressure for 2 hours at 50° C. After confirmation of complete reaction by TCL method, add triethylamine to terminate the reaction, concentrate the mixed solution under reduced pressure at 50° C., and recrystallize it in isopropanol to obtain 45.3 g white solid. The measured value of ESI-MS M/Z value is 282, and the calculated value is 282.3.

Dissolve the said compound (28.2 g) in 250 ml dry DMF, cool the solution to 0° C., add 7.2 g NaH, stir at this temperature for 30 minutes, add 26 ml benzyl bromide dropwise, then rise to room temperature, and stir and react for 2 hours. After confirmation of complete reaction by TCL method, add methanol to quench the remaining NaH, and then concentrate and distill it under reduced pressure. Dissolve the residue in ethyl acetate, wash it with water and saturated salt water, dry it with anhydrous magnesium sulfate, and distill it under reduced pressure. Then, purify it by silica gel (EA/PE=1:3) to obtain 46.4 g white solid product H1. The value of ESI-ESI-MS M/Z is 462.2, and the calculated value is 462.20.

Preparation of H2: Dissolve compound H1 (46.2 g) in 400 ml 80% acetic acid solution, heat up to 70° C., stir at this temperature for 4 hours, and confirm that the reaction is complete by TLC method. Add 400 ml ethyl acetate to the mixed solution, then slowly add saturated sodium bicarbonate aqueous solution for neutralization, wash the organic phase with sodium bicarbonate aqueous solution, water and saturated salt water in turn, dry it with anhydrous sodium sulfate, and concentrate and distill it under reduced pressure to obtain 31.8 g syrup H2. The value of ESI-MS M/Z is 374.2, and the theoretical calculated value is 374.17.

Preparation of H3: Dissolve 29.9 g H2 in 350 ml dry dichloromethane, add 39 g DMAP, cool it to −20° C., add 25 g trifluoromethanesulfonic anhydride dropwise, react for 30 minutes at this temperature, then naturally rise to room temperature and continue to react for 2 hours. After confirming the reaction is complete by TLC method, cool the mixed liquor to −10° C., then add 10 g acetic anhydride dropwise, react for 30 minutes at this temperature, then naturally rise to room temperature and continue to react for 3 hours. After confirming the reaction is complete by TLC method, pour the mixed liquor into precooled saturated sodium bicarbonate solution, and add 200 ml dichloromethane for extraction. Wash the organic phase with saturated sodium bicarbonate, water and 10% salt water in turn, dry it with anhydrous sodium sulphate, filter, and remove the organic solvent by rotary evaporator. Rapidly chromatograph the residue with silica gel column, elute with ethyl acetate/n-hexane (1:2), collect the product, and then remove the organic solvent by rotary evaporator. Dissolve 16 ml ethyl methanesulfonate in 300 ml THF, cool it to −78° C. in a dry N₂ atmosphere, add 64 ml 2.5M n-butyllithium solution and react at this temperature for 30 minutes. Then add the THF solution (100 ml) of the above purified solid dropwise, and allow the mixture to react for 1 hour at this temperature. Then heat up to −20° C., and allow it to react at this temperature until the reaction is confirmed by TLC to be completed. Add an equal volume of saturated ammonium chloride to suspend the reaction, extract with 500 ml ethyl acetate, and wash the organic phase with 150 ml saturated ammonium chloride, water and saturated salt water in turn. Then dry it with anhydrous sodium sulphate, remove the organic solvent by rotary evaporator, dissolve the residue in 200 ml methanol and cool it to 0° C. Then add 3 g sodium methoxide, stir for 2 hours, neutralize with acetic acid, and evaporate out the organic solvent. Chromatograph the residue with silica gel column and eluted with ethyl acetate/n-hexane (1:2). Then collect the product, and remove the organic solvent by rotary evaporator to obtain 25 g white solid H3. ¹H-NMR (600 MHz, CDCl3): δ7.37-7.23 (m, 10H, Ar—H), 4.98 (d, 1H, Ph-CH2), 4.74-4.69 (m, 2H, Ph-CH2), 4.62 (d, 1H, Ph-CH2) 4.54 (d, J=3.4 Hz, 1H, H-1), 4.23 (q, 2H, SO3CH2CH3), 3.72 (t, 1H, H-4), 3.57 (m, 1H, H-5), 3.45 (dd, 1H, H-2), 3.32 (s, 3H, OCH3), 3.29-3.15 (m, 2H, H-3, H-7a), 3.09 (m, 1H, H-7b), 2.40-2.28 (m, 1H, H-6a), 1.95-1.84 (m, 1H, H-6b), 1.34 (t, 3H, SO3CH2CH3).

### EXAMPLE 2

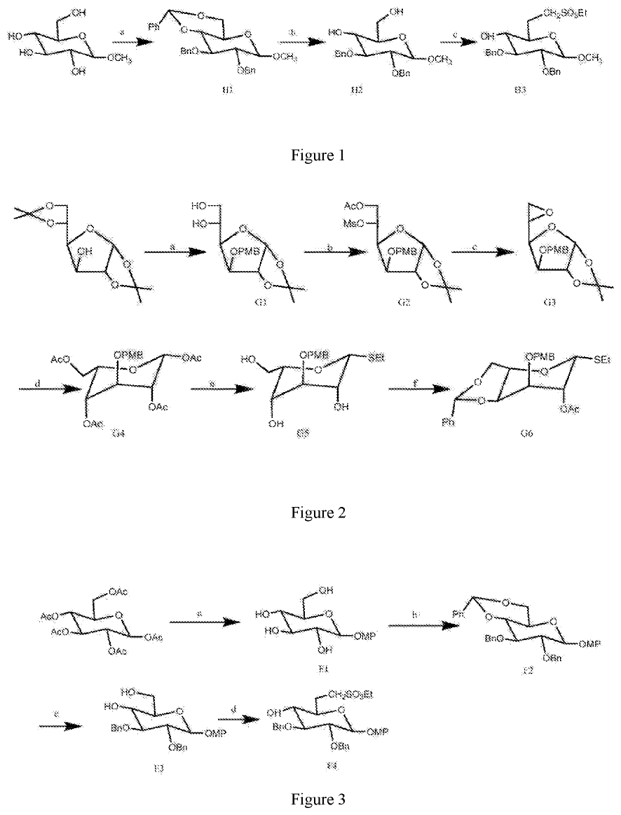

This example provides a method for synthesizing building block G with diacetone glucose, as shown in FIG. 2, and under the following conditions: a) 1. PMBCl, NaH, THF, 60° C.; 2.60% HAc, two-step yield 82%; b) 1. MSC1, Py; 2. KAc, ACN (two-step yield 72%); c) t-BuOK, t-BuOH, 76%; d) 1. 0.1M H₂SO₄; 2. Ac₂O, Py, (two-step yield 76%); e) 1. EtSH, BF₃.Et₂O, CH₂Cl₂; 2. NaOMe, MeOH; f) 1. PhCH(OCH₃)₂, p-TsOH, DMF; 2. Ac₂O, DMAP, e and f two-step yield 72%.

Preparation of G1: dissolve diacetone glucose (52.0 g, 0.2 mol) in tetrahydrofuran (520 ml), cool the solution to 0° C., then add sodium hydride (12.3 g, 1.5 eq), and stir and react at this temperature for 30 minutes. Then, add p-methoxybenzyl chloride (PMBC1, 40.7 ml, 1.5 eq) dropwise. After drop-wise addition, heat up the mixture to 60° C. and stir it for 6 hours. After confirmation of complete reaction by TCL method, quench it with methanol, and concentrate it under reduced pressure. Dissolve the residue in ethyl acetate, wash it with water and saturated salt water, dry it with anhydrous sodium sulfate, and distill it under reduced pressure.

Dissolve the said compound in 60% acetic acid solution (600 ml), stir and react for two days at room temperature. After confirmation of complete reaction by TCL method, distill it under reduced pressure to remove most solvents, and then add 400 ml methylene chloride for extraction. Wash the organic phase with saturated sodium bicarbonate, water and 10% salt water in turn, dry it with anhydrous sodium sulfate, and remove the organic solvent by rotary evaporator to obtain 55.8 g light yellow syrup G1.

Preparation of G2: dissolve compound G1 (34 g) in the mixed solution of pyridine (150 ml) and dichloromethane (200 ml), cool the solution to 0° C., add the pyridine solution of MsCl (2.4 eq, dissolved in 50 ml pyridine) dropwise, and stir overnight. After confirmation of complete reaction by TCL method, pour the mixed solution into 1.5 L warm water and stir to form a light yellow precipitate. After filtration, dry it to obtain a crude product, and then directly carry out the next reaction with it without purification. Dissolve the crude product G3 in acetonitrile (200 ml), and add 55 g anhydrous potassium acetate. After heating and refluxing for 48 h and confirmation of complete reaction by TCL method, filter out the solid, distill the filtrate liquor under reduced pressure, and purify the residue by silica gel column (EA/PE=1:3) to obtain 33.1 g light yellow solid. The measured ESI-MS M/Z value is 460.1, and the theoretical calculated value is 460.14.

Preparation of G3: dissolve compound G3 (23 g) in dichloromethane (250 ml), add tert-butyl alcohol (80 ml) and potassium tert-butoxide (10 g) at 0° C., and stir overnight at this temperature under the protection of nitrogen. After confirmation of complete reaction by TCL method, filter through diatomite filter plate, concentrate the filtrate liquor under reduced pressure, and purify the residue by silica gel (EA/PE=1:2->1:1) to obtain 11.9 g yellow syrup G3.

Preparation of G4: dissolve compound G4 (9.7 g) with 50 ml ethanol, add 0.1M sulfuric acid solution (200 ml), and then stir and react for 16 hours at 60° C. After confirmation of complete reaction by TCL method, cool it to room temperature, add barium carbonate to neutralize to pH=8, filter, and concentrate and distill the filtrate liquor under reduced pressure. Add ethanol and toluene to the residue, repeatedly distill to remove water and obtain light yellow syrup which is directly used for the next reaction without purification.

Add pyridine (100 ml) to the said product, stir and dissolve, then cool the solution to 0° C., add acetic anhydride (55 ml), stir at this temperature for 1 hour, then heat up the mixed solution to room temperature, and continue to stir overnight. After confirmation of complete reaction by TCL method, add methanol for quenching, and then distill and concentrate it under reduced pressure. Dissolve the residue in ethyl acetate, wash it with 5% sodium bisulfate aqueous solution, water, sodium bicarbonate aqueous solution, water and saturated salt water in turn, dry it with anhydrous sodium sulfate, and distill it under reduced pressure. Purify the residue by silica gel (EA/PE=1:3) to obtain 10.7 g white solid G4. The measured value of ESI-MS M/Z is 468.2, and the theoretical calculated value is 468.16.

Preparation of G5: dissolve compound G4 (9.4 g) in anhydrous dichloromethane (100 ml), add ethanethiol (1.5 g, 1.2 eq), then cool down to 0° C., and add boron trifluoride ether solution (4.1 ml, 1.5 eq, dissolved in 20 ml dichloromethane) dropwise. After drop-wise addition, react at this temperature for 1 hour, naturally rise to room temperature, and continue to stir for 4 hours. After confirmation of complete reaction by TCL method, add saturated sodium bicarbonate solution to neutralize to pH=7. Wash the organic phase with sodium bicarbonate solution, water and saturated salt solution in turn, dry it with anhydrous sodium sulfate, and distill and concentrate it under reduced pressure. Dissolve the residue in methanol (100 ml), add sodium methoxide (1.0 g) and stir for 3 hours at room temperature. After confirmation of complete reaction by TCL method, neutralize it with Dow acid resin and distill and concentrate it under reduced pressure to obtain light yellow syrup G5 which is directly used for the next reaction without purification.

Preparation of G6: dissolve crude product G5 in THF (100 ml), add p-toluenesulfonic acid monohydrate (1.2 g) and benzylidene reagent PhCH (OMe)₂ (5 ml), heat up to 60° C., and react for 4 h under vacuum. After confirmation of complete reaction by TCL method, cool down to room temperature, add triethylamine for neutralization, and distill and concentrate it under reduced pressure. Dissolve the residue in dichloromethane (100 ml), and add 3.5 g DMAP. After dissolution, cool it to 0° C., and add acetic anhydride (3.2 ml) dropwise. After stirring at this temperature for 1 hour, naturally rise to room temperature, and continue to stir overnight. After confirmation of complete reaction by TCL method, pour it into the precooled saturated sodium bicarbonate aqueous solution to separate the organic phase while use 100 ml dichloromethane to extract the aqueous phase three times, and then combine the organic phases, wash it with water and saturated salt water in turn, dry it with anhydrous sodium sulfate, and distill it under reduced pressure. Purify the residue by silica gel (EA/PE=1:4) to obtain 7.0 g white solid G6. The measured value of ESI-MS M/Z is 474.2, and the theoretical calculated value is 474.17. ¹H-NMR (600 Hz, CDCl₃): δ7.43-7.26 (m, 5H, Ar—H), 7.01-6.91 (m, 4H, Ar—H), 5.98 (s, Ph-CH), 5.24 (d, 1H, H-1), 4.87 (m, 1H, H2), 4.63 (s, 2H, CH30-Ph-CH2), 4.18-4.10 (m, 2H, H-3, H-4), 4-05 (m, 1H, H-6a), 3.87 (m, 1H, H-6b), 3.81 (s, 3H, CH3OPh), 2.48 (m, 2H, SCH2CH3), 2.01 (s, 3H, CH3CO), 1.1 (t, 3H, SCH2CH3).

### EXAMPLE 3

This example provides a method for synthesizing building block F with β-pentaacetyl glucose, as shown in FIG. 3, and the conditions are as follows: a) 1. 4-Methoxyphenol (MPOH), BF₃.Et₂O; 2. NaOMe, MeOH, two-step yield 89%; b) 1. PhCH(OMe)₂, p-TsOH; 2. NaH, BnBr, DMF, 83%; c) 80% HAc, 70° C., 86%; d) 1. Tf₂O, DMAP; 2. Ac₂O, DMAP; 3. BuLi, CH₃SO₃Et, −20° C.; 4. CH₃ONa, CH₃OH; four-step yield 64%.

Preparation of F1: dissolve β-pentaacetyl glucose (39 g) in dry dichloromethane (250 ml), add p-methoxyphenol (16.1 g), cool the mixture to 0° C., add boron trifluoride ether solution (18.8 ml, dissolved in 50 ml dichloromethane) dropwise, stir at this temperature for 30 minutes, and then rise to room temperature to continue the reaction for 4 hours. After confirmation of complete reaction by TCL method, add saturated sodium bicarbonate to neutralize, use ethyl acetate to extract, and wash the organic phase with saturated sodium bicarbonate solution, water and saturated salt solution, dry it with anhydrous magnesium sulfate, and distill and concentrate it under reduced pressure. Dissolve the residue in methanol (250 ml), and add sodium methoxide (2.5 g) at room temperature. After stirring for reaction for 14 hours and confirmation of complete reaction by TCL method, neutralize with Dow acid resin, and distill and concentrate it under reduced pressure to obtain a light pink solid. After silica gel column chromatography (EA/n-hexane=1:1-2:1), a light yellow syrup F1 (25.4 g) is obtained.

Preparation of F2: dissolve 14.3 g F2 in THF (150 ml), add benzylidene reagent PhCH (OMe)₂ (8 ml) and p-toluenesulfonic acid monohydrate (1.5 g), and heat up to 60° C. and react for 4 hours under vacuum. After confirmation of complete reaction by TCL method, cool the mixture to room temperature, add triethylamine for neutralization, and then distill and concentrate it under reduced pressure. Recrystallize the residue with isopropanol/n-hexane, filter and dry it to obtain white solid. Dissolve the said solid in dry DMF (250 ml), cool it to 0° C. under the protection of nitrogen, and add sodium hydride (6.0 g, 60% sodium hydride/heavy oil). After stirring at this temperature for 30 minutes, add benzyl bromide (15 ml) dropwise and continue to stir for 4 hours. After confirmation of complete reaction by TCL method, add methanol for quenching, pour the mixed solution into 2 L water, stir to produce white solid, filter, wash with petroleum ether and dry it to obtain 23.1 g white solid F2. The measured value of ESI-MS M/Z is 554.2, and the theoretical calculated value is 554.23.

Preparation of F3: Dissolve Compound F2 (16.6 g) in 80% acetic acid (200 ml), heat up to 70° C., and stir and react for 12 hours. After confirmation of complete reaction by TCL method, cool it down, concentrate by rotary evaporation, add 200 ml dichloromethane into the residue, wash it with saturated sodium bicarbonate, water and 10% salt water in turn, dry it with anhydrous sodium sulfate, remove the organic solvent by rotary evaporator, and then after silica gel column chromatography (ethyl acetate/n-hexane=1:1-2:1) on the residue, collect the product, and then remove the organic solvent by rotary evaporator to obtain 11.6 g product F3. The measured value of ESI-MS M/Z is 466.2, and the theoretical calculated value is 466.20.

Preparation of F4: Dissolve 9.3 g F3 in 100 ml dry dichloromethane, add 8.0 g DMAP, cool down to −20° C., drop 6.2 g trifluoromethanesulfonic anhydride dropwise, react at this temperature for 30 minutes, then naturally rise to room temperature, and continue to react for 2 hours. After confirmation of complete reaction by TCL method, cool the mixed solution to 0° C., add 2.5 g acetic anhydride dropwise, and react at this temperature for 30 minutes, naturally rise to room temperature and continue to react for 3 hours. After confirmation of complete reaction by TCL method, pour the mixed solution into the precooled saturated sodium bicarbonate solution, add 100 ml dichloromethane for extraction. Wash the organic phase with saturated sodium bicarbonate, water and 10% salt water in turn, dry it with anhydrous sodium thioate, filter and remove the organic solvent by rotary evaporator. After quick silica gel column chromatograph of the residues, elute with ethyl acetate/n-hexane (1:2), collect the product and remove the organic solvent by rotary evaporator. Dissolve 8 ml ethyl methanesulfonate in 150 ml THF, cool down to −78° C. in dry N₂ atmosphere, add 32 ml 2.5M n-butyllithium solution, react at this temperature for 30 minutes, then add the THF (50 ml) solution of the said purified solid dropwise, allow the mixture to react at this temperature for 1 hour, then heat up to −20° C., and react at this temperature until confirmation of complete reaction by TCL method. Add the same volume of saturated ammonium chloride to stop the reaction, extract with 250 ml ethyl acetate, wash the organic phase with 100 ml saturated ammonium chloride, water and saturated salt water in turn, dry it with anhydrous sodium sulfate, remove the organic solvent by rotary evaporator, dissolve the residue in 100 ml methanol and cool down to 0° C., add 1.5 g sodium methoxide, stir for 2 h, neutralize with acetic acid, and evaporate out the organic solvent. After silica gel column chromatography of the residue, elute with ethyl acetate/n-hexane (1:2), collect the product, and remove the organic solvent by rotary evaporator to obtain 7.3 g white solid. F4. ¹H-NMR (600 MHz, CDCl3): δ7.37-7.23 (m,14H,Ar—H), 4.98-4.83 (m,2H,Ph-CH2), 4.74-4.65 (m,2H,Ph-CH2), 4.56 (d, J=3.4 Hz,1H,H-1), 4.23 (q,2H,SO3CH2CH3), 3.70 (t,1H,H-4), 3.58 (m,1H,H-5), 3.44 (dd,1H,H-2), 3.30 (s,3H,0CH3), 3.29-3.15 (m,2H,H-3,H-7a), 3.09(m,1H,H-7b), 2.40-2.28 (m, lH,H-6a), 1.95-1.84 (m, 1H,H-6b), 1.34 (t,3H,SO3CH2CH3).

### EXAMPLE 4

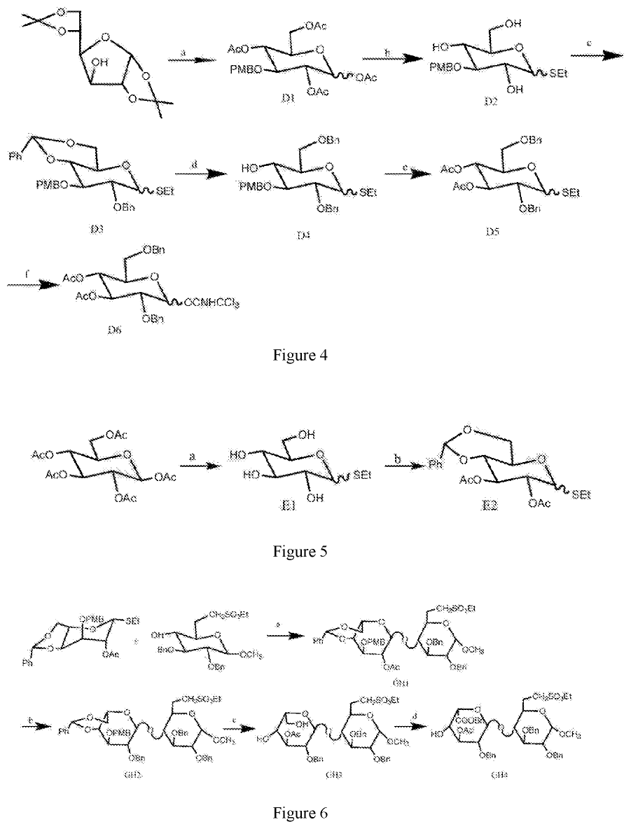

This example provides a method for synthesizing building block F with diacetone glucose, as shown in FIG. 4, and under the following conditions: a) 1. PMBCl, NaH, THF, 60° C.; 2. 0.2M H₂SO₄, 60° C.; 3. Ac₂0, Pyridine (three-step yield 69%); b) 1. EtSH, BF₃.Et₂0, CH₂CI₂, 0° C.; 2. NaOMe, MeOH (two-step yield 82%); c) 1. PhCH (OMe)₂, p-TsOH, THF, 70° C.; 2. NaH, BnBr, THF (two-step yield 81%); d) Et₃SiH, TFA, TFAA, CH₂CI₂, 0° C. (84%); e) 1. (NH₄)₂Ce (N0₃)₆, CH₂CI₂/H₂O; 2) Ac₂O, Pyridine (two-step yield 67%); f) 1. NIS, TMSOTf, Acetone/H₂0; 2. CCl₃CN, K₂CO₃, CH₂Cl₂ (two-step yield 63%).

Preparation of D1: put 3-0-p-methoxybenzyl diacetone glucose (refer to G1) (76 g) in 300 ml 0.2M sulfuric acid solution, heat up to 60° C., and stir and react for 18 h. After confirmation of complete reaction by TCL method, add excessive BaCO₃ to neutralize it, filter to remove the solid, then remove water from the solution by rotary evaporator, and then remove a small amount of water by evaporate it with ethanol and toluene. Add 300 ml pyridine to the residue, stir and dissolve, then cool the solution to −10° C., add 100 ml acetic anhydride dropwise, and then continue to react at this temperature for 2 hours. Naturally rise to room temperature and react overnight. Pour the mixture into ice water, extract it three times with 300 ml ethyl acetate, and combine the organic phases. Wash the organic phases with 150 ml of dilute hydrochloric acid, water, saturated sodium bicarbonate aqueous solution and saturated salt water in turn, dry it with anhydrous sodium sulfate, and remove the organic solvent by rotary evaporator. Recrystallize the residue with ethyl alcohol, filter, dry the solid to obtain the white solid D1 (64.6 g). The measured value of ESI-MS M/Z is 468.2, and the theoretical calculated value is 468.16.

Preparation of D2: Dissolve D1 (46.8 g) in dry dichloromethane (500 ml), add ethanethiol (10.2 ml), cool the mixed solution to 0° C. under the protection of argon, add boron trifluoride ether solution (19.8 ml, dissolved in 50 ml dichloromethane) dropwise, and stir at this temperature for 4 hours. After confirmation of complete reaction by TCL method, add saturated sodium bicarbonate solution to neutralize to pH=7-8. Wash the organic phase with saturated sodium bicarbonate aqueous solution, water and saturated salt water, dry it with anhydrous sodium sulfate, and distill and concentrate it under reduced pressure. Dissolve the residue in methanol (400 ml), add sodium methoxide (4 g) at room temperature, stir and react for 6 hours, then neutralize it with Dow acidic resin, and distill it under reduced pressure to obtain light yellow syrup. After silica gel column chromatography (ethyl acetate/n-hexane), collect the product, remove the organic solvent by rotary evaporator to obtain syrup D2 (28.2 g). The measured value of ESI-MS M/Z is 344.1, and the theoretical calculated value is 344.13.

Preparation of D3: Add THF (200 ml), benzylidene reagent PhCH (OMe)₂ (12 ml) and p-toluenesulfonic acid (1.5 g) to D2 (17.2 g), heat up to 70° C., react for 3 hours, neutralize with triethylamine, and concentrate it under reduced pressure. Recrystallize the residue with isopropanol/petroleum ether to obtain a white solid. Dissolve it in THF (150 ml), cool down to 0° C. under the protection of argon, and add NaH (3 g, 60%). Stir at this temperature for 30 minutes, then add benzyl bromide (6.7 ml) dropwise and continue to stir for 3 hours. After confirmation of complete reaction by TCL method, add methyl alcohol for quenching, pour the mixed solution into water, form white solid precipitate, recrystallize the solid with ethyl acetate/n-hexane, and dry it to obtain compound D3 (21.2 g). The measured value of ESI-MS M/Z is 522.2, and the theoretical calculated value is 522.21.

Preparation of D4: Dissolve compound D3 (15.7 g) in dry dichloromethane (144 ml), add triethylsilane (19.2 ml), and cool down to 0° C. under argon protection. Then, add a mixed solution of trifluoroacetic acid (9.3 ml) and trifluoroacetic anhydride (0.7 ml) dropwise, and after the drop-wise addition is completed, continue to stir at this temperature for 4 hours, and confirm complete reaction by TCL method. Add 220 ml ethyl acetate to the mixed solution, then slowly add 4N sodium hydroxide solution (33 ml), and then adjust the pH value of the mixed solution to 8 with sodium bicarbonate solution. Wash the organic phase with aqueous sodium bicarbonate solution, water and saturated salt water, dry it with anhydrous sodium sulfate, and distill and concentrate it under reduced pressure. Purify the residue by silica gel (EA/PE=1:2) to obtain colorless syrup D4 (13.2 g). The measured value of ESI-MS M/Z is 524.2, and the theoretical calculated value is 524.22.

Preparation of D5: Dissolve compound D4 (13 g) in dichloromethane/water (150 ml, CH₂Cl₂/H₂O=20/1), add CAN (40.7 g), and stir overnight at room temperature. After confirmation of complete reaction by TCL method, add dichloromethane (150 ml), wash the organic phase with saturated aqueous solution of sodium bicarbonate, water and saturated salt water in turn, dry it with anhydrous sodium sulfate, and distill it under reduced pressure. Directly use the residue for the next reaction without purification. Add pyridine (100 ml) to the said residue, dissolve it, cool down to 0° C., add acetic anhydride (40 ml), stir at this temperature for 1 hour, then rise to room temperature, and stir for reaction overnight. After confirmation of complete reaction by TCL method, add water for quenching, distill and concentrate it under reduced pressure, and purify the residue by silica gel to obtain 8.1 g colorless syrup D5. The measured value of ESI-MS M/Z is 488.2, and the theoretical calculated value is 488.19.

Preparation of D6: Dissolve compound D5 (5 g) in acetone/water (50 ml, Acetone/H₂0=12/1), cool down to −10° C., add NIS (3.9 g), then add a small amount of TMSOTf dropwise, and continue to stir until complete reaction is confirmed by TCL method. Add sodium thiosulfate/sodium bicarbonate aqueous solution to terminate the reaction, extract with ethyl acetate, wash the organic phase with water and saturated salt solution, dry it with anhydrous sodium sulfate, distill it under reduced pressure, and directly use the residue for the next reaction without purification. Dissolve the said crude product in dry dichloromethane (50 ml), add trichloroacetonitrile (4.5 ml), and then add anhydrous potassium carbonate (2.5 g). Stir at room temperature for 3 hours, filtrate, and distill the filtrate liquor under reduced pressure. Purify the residue by silica gel (EA/PE=1:3, adding 1% triethylamine) to obtain 3.8 g white solid. The measured value of ESI-MS M/Z is 588.1, and the theoretical calculated value is 588.09.

### EXAMPLE 5

The example provides a method for synthesizing building block E with pentaacetyl glucose, as shown in FIG. 5, and the conditions are as follows: a) 1. EtSH, BF₃Et₂0, CH₂Cl₂, 0° C.; 2. NaOMe, MeOH (two-step yield 82%); b) 1. PhCH (OMe)₂, p-TsOH, DMF, 60° C.; 2. AC₂0, DMAP, CH₂Cl₂, 0° C. (two-step yield 78%).

Preparation of E1: Dissolve β-pentaacetyl glucose (39 g) in dry dichloromethane (500 ml), add ethanethiol (10.2 ml), cool the mixed solution to 0° C. under the protection of argon, add boron trifluoride ether solution (19.8 ml, dissolved in 50 ml dichloromethane) dropwise, and stir at this temperature for 4 hours. After confirmation of complete reaction by TCL method, add saturated sodium bicarbonate solution to neutralize to pH=7-8. Wash the organic phase with saturated sodium bicarbonate aqueous solution, water and saturated salt water, dry it with anhydrous sodium sulfate, and distill and concentrate it under reduced pressure. Dissolve the residue in methanol (400 ml), add sodium methoxide (4 g) at room temperature, stir and react for 6 hours, then neutralize it with Dow acidic resin, and distill it under reduced pressure to obtain light yellow syrup. After silica gel column chromatography (ethyl acetate/n-hexane=1:1-2:1), collect the product, remove the organic solvent by rotary evaporator to obtain syrup E1 (18.4 g). The measured value of ESI-MS M/Z is 224.1, and the theoretical calculated value is 224.07.

Preparation of E2: add THF (200 ml), benzylidene reagent PhCH (OMe)₂ (12 ml) and p-toluenesulfonic acid (1.5 g) in E1 (11.2 g), heat up to 70° C., react for 3 hours, neutralize with triethylamine, and concentrate it under reduced pressure. Recrystallize the residue with isopropanol/petroleum ether to obtain a white solid. Dissolve the solid in dichloromethane (100 ml), add DMAP (18.5 g), and after dissolution, cool the mixed solution to −10° C., add acetic anhydride (16 ml) dropwise, react at this temperature for 1 hour, then naturally rise to room temperature, and continue to react for 3 hours. Pour the solution into pre-cooled saturated sodium bicarbonate aqueous solution, and extract with dichloromethane (100 ml). Wash the organic phase with saturated sodium bicarbonate, water and 10% salt solution in turn, dry it with anhydrous sodium sulfate, remove the organic solvent by rotary evaporator, and recrystallize it with ethyl acetate/n-hexane to obtain a white solid that is the product E2 (15.5 g) after drying. The measured value of ESI-MS M/Z is 396.1, and the theoretical calculated value is 396.12.

### EXAMPLE 6

The example provides a method for synthesizing building block disaccharide GH with building blocks G and H, as shown in FIG. 6, and under the following conditions: a) NIS, AgOTf, Toluene, 0° C., 85%; b) 1. NaOMe, MeOH; 2) NaH, BnBr and THF (two-step yield 92%); c) 60% HAc, 85%; d) 1. DAIB, TEMPO, ACN/H₂O; 2. BnBr, K₂CO₃ (two-step yield 82%).

Preparation of GH1: dissolve compound G6 (6.2 g) and compound H3 (4.8 g) in toluene (100 ml), add newly activated 4 A molecular sieve (5 g) and stir for 30 min at room temperature. Under the protection of argon, cool the mixture to 0° C., add NIS (4.6 g), and stir at this temperature for 10 minutes. Then, add AgOTf solution (0.62 g, dissolved in 20 ml toluene) dropwise, and continue to stir until the reaction is complete. After filtering out the molecular sieve, wash it to light yellow with sodium thiosulfate/sodium bicarbonate aqueous solution, then wash it with water and saturated salt water, dry it with anhydrous sodium sulfate, distill it under reduced pressure, and purify the residue by silica gel to obtain 7.6 g white solid GH1. The measured value of ESI-MS M/Z is 894.4, and the theoretical calculated value is 894.35.

Preparation of GH2: dissolve compound GH1 (6.1 g) in methanol (80 ml), add sodium methoxide (1 g) at room temperature, and stir until the reaction is complete. After neutralization with Dow acidic resin, distill and concentrate it under reduced pressure, then without purification, directly use it for the next reaction. Dissolve the said crude product in THF (100 ml), cool down to 0° C. under argon protection, add NaH (0.4 g, 60%), and after stirring at this temperature for 30 minutes, add benzyl bromide (1.2 ml) dropwise. Continue to stir until the reaction is confirmed by TCL method to be completed. Add methanol for quenching, then distill and concentrate the mixed solution under reduced pressure, add ethyl acetate to the residue, wash it with water and saturated salt water, dry it with anhydrous sodium sulfate and distill it under reduced pressure. Purify the residue by silica gel to obtain 5.9 g white solid GH2. The measured value of ESI-MS M/Z is 942.4, and the theoretical calculated value is 942.39.

Preparation of GH3: dissolve compound GH2 (4.7 g) in 60% acetic acid solution (20 ml), stir and react for 4 h at 60° C., then distill it under reduced pressure, and purify the residue by silica gel (ethyl acetate/n-hexane=1:1) to obtain 3.9 g colorless syrup GH3.

Preparation of GH4: Dissolve compound GH3 (3.5 g) in acetonitrile/water (35 ml, ACN/H₂O=1:1), add TEMPO (0.1 g) and iodobenzene diacetate (DIAB, 2.2 g) at room temperature, stir vigorously for 3 hours, then add methanol for quenching, and distill and concentrate the residue under reduced pressure. Without purification, directly use the residue in the next reaction. Dissolve the said crude product in DMF (20 ml), add benzyl bromide (2 ml) and anhydrous potassium carbonate (5 g), and stir overnight at room temperature. After confirmation of complete reaction by TCL method, filter it on a diatomite filter plate, distill and concentrate the filtrate liquor under reduced pressure. Add ethyl acetate into the residue, wash it with water and saturated saline solution, dry it with anhydrous sodium sulfate, and distill it under reduced pressure. Purify the residue by silica gel (ethyl acetate/n-hexane=1:2) to obtain 3.25 g white solid GH4. The measured value of ESI-MS M/Z is 880.3, and the theoretical calculated value is 880.33. ¹H-NMR (600 MHz, CDCl₃): δ7.15-7.40 (m,20H,Ar—H), 5.62 (d,1H,H-1), 5.34-5.36 (m,2H,C00CH2Ph), 4.97 (m,1H,G-3), 4.92 (m,1H,G-4), 4.61-4.64(m,6H,Ph-CH2), 4.54(d,1H,G-1), 4.43 (d,1H,G-5), 4.23 (q,2H,SO3CH2CH3), 3.45-3.84 (m,3H, H-2,H-4,H-5), 3.38 (s,0CH3), 3.29-3.15 (m,2H,H-3,H-7a), 3.09 (m,1H,H-7b), 2.40-2.28(m,1H,H-6a), 1.95-1.84 (m, 1H,H-6b), 1.34 (t,3H,SO3CH2CH3).

### EXAMPLE 7

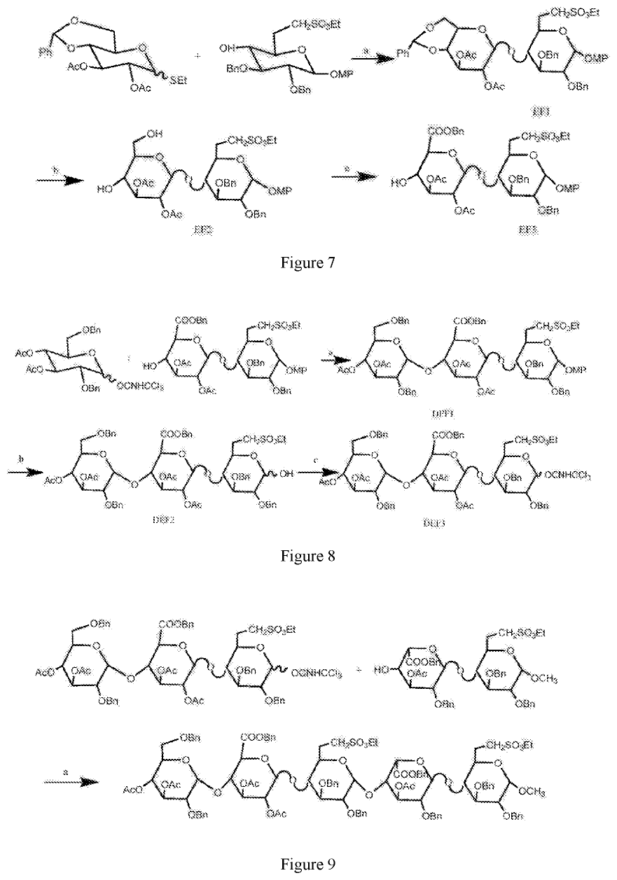

The example provides a method for synthesizing disaccharide building block EF with building block E and building block F, as shown in FIG. 7, and the conditions are as follows: a) NIS, AgOTf, Toluene, 0° C., 83%; b) 60% HAc, 85%; e) 1. DAIB, TEMPO, ACN/H₂O; 2. BnBr, K₂CO₃ (two-step yield 83%).

Preparation of EFL: dissolve E2 (25.8 g) and monosaccharide building block F4 (28.7 g) in dry toluene (550 ml), add newly activated 4 A molecular sieve (30 g), stir at room temperature for 30 minutes, cool the mixture to −5° C. In nitrogen atmosphere, add NIS (28. Lg) and add AgOTf solution (2.5 g dissolved in 50 ml toluene) dropwise. Continue to stir at this temperature for 1 hour, then naturally rise to the room temperature, and continue to react for 2 hours. After confirmation of complete reaction by TCL method, filter out the molecular sieve, then wash it to light yellow with sodium thiosulfate/sodium bicarbonate solution, then wash it with water and saturated salt water, dry it with anhydrous sodium sulfate, and distill it under reduced pressure. Purify the residue by silica gel to obtain 38.6 g white solid EF1. The measured value of ESI-MS M/Z is 908.3, and the theoretical calculated value is 908.33.

Preparation of EF2: dissolve compound EF1 (27.3 g) in 60% acetic acid aqueous solution, heat up to 60° C., and stir and react for 12 hours. After confirmation of complete reaction by TCL method, distill and store it under reduced pressure. Purify the residue by silica gel (ethyl acetate/n-hexane) to obtain 21.0 g colorless syrup EF2.

Preparation of EF3: dissolve compound EF2 (16.4 g) in acetonitrile/water (200 ml, ACN/H20=3:1), add TEMPO (0.4 g) and iodobenzene diacetate (DIAB, 11.5 g) at room temperature, vigorously stir and react for 3 hours, add methanol for quenching, distill and concentrate it under reduced pressure, and distill 60 ml toluene three times in mixed way. Directly dissolve the residue in DMF (150 ml) without purification, add benzyl bromide (10.5 ml) and anhydrous potassium carbonate (28 g), and stir overnight at room temperature. After confirmation of complete reaction by TCL method, filter it on a diatomite filter plate, and distill the filtrate under reduced pressure. Add Ethyl acetate (300 ml) to the residue, wash it with water and saturated salt water, dry it with anhydrous sodium sulfate, and distill it under reduced pressure. Purify the residue by silica gel to obtain 15.3 g white solid EF3. The measured value of ESI-MS M/Z is 924.3, and the theoretical calculated value is 924.32. ¹H-NMR(600 MHz, CDCl3), δ7.31-7.47 (m, 15H,Ar—H), 6.84-6.98 (m,4H,Ph-0CH3), 5.74 (d, 1H,F-1), 5.34-5.42 (m, 2H, C00CH2Ph) 5.24 (m, 1H,E-2), 4.92-4.74 (m, 2H, E-3,E-4), 4.45-4.64 (m,5H,E-1,Ph-CH2), 4.21 (q,2H,SO3CH2CH3), 3.45-3.84 (m,3H,F-2,F-4,F-5), 3.80 (s,OCH3), 3.29-3.15 (m,2H,F-3,F-7a), 3.09 (m, 1H,F-7b), 2.40-2.28 (m, 1H,H-6a), 1.95-1.84 (m,1H,H-6b), 1.34 (t,3H,SO3CH2CH3).

### EXAMPLE 8

The example provides a method for synthesizing trisaccharide building block DEF with building block D6 and disaccharide building block EF3, as shown in FIG. 8, and under the following conditions: a) TBSOTf, CH₂Cl₂, −20° C., 78%; b) CAN, ACN/H₂O, 80%; c) CCl₃CN, K₂CO₃, CH₂Cl₂, 76%.

Preparation of DEF1: dissolve building block D6 (36.8 g) and disaccharide building block EF3 (46.2 g) in dry dichloromethane (800 ml), add newly activated 4 A molecular sieve (40 g), stir at room temperature for 30 min, then cool the mixture to −20° C., add TBSOTf (6.5 ml, dissolved in 100 ml dichloromethane) dropwise, stir at this temperature for 30 minutes, then rise to room temperature, stir and react for 2 hours, and neutralize it with triethylamine. Filter the mixed solution with diatomite filter plate, wash the filtrate liquor with water and saturated salt water, dry it with anhydrous sodium sulfate, and distill it under reduced pressure. Purify the residue by silica gel (EA/PE=1/3) to obtain 52.7 g white solid DEF1. The measured value of ESI-MS M/Z is 1350.5, and the theoretical calculated value is 1350.49.

Preparation of DEF2: dissolve compound DEF1 (40.5 g) in acetonitrile/toluene (500 ml, ACN/Toluene=1.5/1), cool the mixed solution to 0° C., and slowly add cerium (IV) ammonium nitrate (60 g, dissolved in 250 ml water), stir at this temperature for 30 min, add ethyl acetate to the mixed solution to dilute it. Wash the organic phase with saturated sodium bicarbonate solution and saturated salt water, dry it with anhydrous sodium thiosulfate, and distill and store it under reduced pressure. Purif the residue by silica gel (EA/PE=1/2-1/1) to obtain 29.8 g light yellow syrup DEF2. The measured value of ESI-MS M/Z is 1244.5, and the theoretical calculated value is 1244.45. ¹H-NMR: δ7.41-7.26 (m,25H,Ar—H), 5.81 (d,1H,D-1), 5.65(d,1H,F-1), 5.15-5.20 (m, 2H, COOCH2Ph), 4.65-4.98(m,15H), 4.51(d,1H,E-1), 4.23 (q,2H,SO3CH2CH3), 3.60-3.93 (m,6H), 3.35(m,1H), 3.29-3.15 (m,2H), 3.07(m,1H,H-7b), 2.40-2.28(m,1H), 2.01 (s,12H), 1.95-1.84 (m, 1H,H-6b), 1.34 (t,3H,SO3CH2CH3).

Preparation of DEF3: dissolve the compound DEF2 (25 g) in dry dichloromethane (300 ml), add trichloroacetonitrile (12 ml), then add anhydrous potassium carbonate (25 g), stir at room temperature for 3 h, filter, and distill the filtrate liquor under reduced pressure. Purify the residue by silica gel (EA/PE=1:3-1/2) to obtain 21.1 g white foamy solid DEF3.

### EXAMPLE 9

The example provides a method for synthesizing fully-protected pentose with trisaccharide building block DEF3 and disaccharide building block GH4, as shown in FIG. 9, and under the following conditions: a) TMSOTf, CH₂Cl₂, −20° C., 75%.

Preparation of Pentose DEFGH: dissolve trisaccharide building block DEF3 (18.1 g) and disaccharide building block GH5 (8.8 g) in dry dichloromethane (250 ml), add newly activated 4 A molecular sieve (15 g), stir at room temperature for 30 min, then cool the mixture to −20° C., and add TMSOTf solution (0.8 ml dissolved in 10 ml dichloromethane) dropwise. After confirmation of complete reaction by TCL method, add triethylamine for neutralization, filter, wash the filtrate liquor with water and saturated salt, dry it with anhydrous sodium sulfate, and distill it under reduced pressure. Purify the residue by silica gel (EA/PE=1/4-1/2) to obtain 15.8 g foamy white solid DEFGH. The measured value of ESI-MS M/Z is 2106.8, and the theoretical calculated value is 2106.77. ¹H-NMR (600 Hz, CDCI3): δ7.18-7.37 (m,45H), 5.56(d,1H), 5.25(d,1H), 5.17(d,1H), 4.83-5.01(m,16H), 4.24-4.30(m,2H), 4.01 (t,1H), 3.85 (s,3H), 3.59-3.84 (m, 10H), 3.29-3.57(m,6H), 3.09-3.25 (m,6H), 2.19-2.41 (m,2H), 2.01-2.03 (s, 15H), 1.78-1.94 (m, 2H), 1.4(t,6H). ¹³C-NMR: δ170.2, 169.3, 169.2, 168.0, 138.9, 138.6, 137.8, 137.6, 137.5, 128.1-126.8, 101.4, 98.0, 97.5, 97.3, 96.8, 83.6, 82.9, 82.3, 81.2, 80.0, 79.5, 79.0, 78.9, 78.6, 78.2, 76.1, 74.5, 74.1, 72.5, 70.7, 68.8, 68.5, 67.7, 67.1, 66.2, 60.5, 60.2, 58.9, 58.4, 58.1, 55.4, 52.2, 46.3, 45.8, 25.8, 25.4, 20.9, 20.7, 15.1.

### EXAMPLE 10

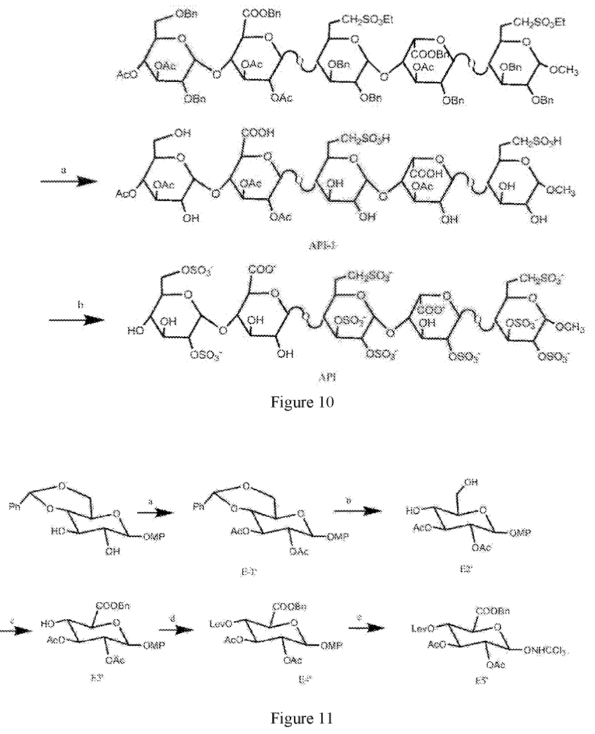

The example provides a method for synthesizing API with fully-protected pentose, as shown in FIG. 10, and under the following conditions: a) 1. Nal, Aceton, RT; 2. 10% Pd/C, H₂; b) SO₃.Et₃N, DMF, 55° C., NaOH.

Preparation of API-1: dissolve fully-protected pentose (4.2 g) in acetone (40 ml), add sodium iodide (1.3 g), react for 24 hours at room temperature, then concentrate it, purify it by Sephadex LH-20 (methanol as eluent), remove the solvent by rotary evaporation, then dissolve it with ethanol/acetic acid (29/1,100 ml), and add 10% Pd/C (2 g). Introduce hydrogen, stir and react at 50° C. for 2 days, filter with 0.45 wn membrane, and distill the filtrate liquor under reduced pressure to obtain crude product residue, which is directly used for the next reaction without purification.

Preparation of AP2: dissolve crude API-1 in dry DMF (50 ml), then add sulfur trioxide triethylamine compound (10 g) at room temperature, rise to 55° C. under argon protection, and stir and react for 24 hours. Cool down to room temperature, slowly add it into the water solution of sodium bicarbonate, adjust the pH value of the mixed solution to 11 with 2M sodium hydroxide, continue to stir and react for 2 hours, adjust the solution to neutrality with ammonium acetate solution after HPLC detection, and desalt with G25 to obtain crude API. After purifying the crude API by ion exchange column (MonoQ), desalt it with G25 again, freeze and dry it to obtain 1.6 g refined API (pentose). The yield of API from fully-protected pentose is 45%. ¹H-NMR (600 Hz, D₂O): δ5.48 (d,J(H1,H2)=3.2 Hz, 1H,D-1), 5.41 (d,J(H1,H2)=2.6 Hz, 1H,F-1), 5.26 (d,J(H1,H2)=2.3 Hz, 1H,G-1), 5.13 (d, J (H1,H2)=3.3 Hz,1H,H-1), 4.86 (d,1H,G-5), 4.68(d,J(H1,H2)=7.7 Hz, 1H,E-1), 4.62(t,H-3), 4.56 (t,1H,F-3), 4.37 (dd,H-2), 4.27-4.33 (m,2H,F-2,D-6a), 4.13-4.15 (m,2H,D-6b,G-4), 4.03 (t,1H,F-5), 3.88-3.94 (m,3H,D-5,E-4,H-5), 3.75-3.84 (m,4H,E-5,H-4,F-4,G-3), 3.65 (s,3H,0CH3), 3.55-3.61 (m,3H,G-2,E-3,D3), 3.29-3.36 (m,3H,E-2,D-2,D-4), 2.98-3.02(m,4H,F-7a,F-7b,H-7a,H-7b),2.37-2.44(m,2H,F-6a,H-6a),1.96-2.03(m,2H,F-6b,H-6b). ¹³C-NMR (150 Hz,D₂0), δ175.2, 175.0 (2C0), 102.8 (E-1), 101.2 (GT), 97.9 (H-1), 96.9 (D-1), 94.5 (F-1), 86.6 (E-3), 83.8 (E-2), 82.6 (D-3), 81.4(D-2), 79.4, 78.8, 77.8, 76.5 (E-5,H-4,F-4,G-3), 78.9 (D-4), 75.1 (E-4), 73.7 (G-4), 71.4 (G-5), 71.0 (F-5), 70.3, 69.7 (H-5,D-5), 66.9 (D-6), 60.8 (OCH3), 48.1, 48.0 (F-7,H-7), 27.2, 27.0 (F-6,H-6).

### EXAMPLE 11

The example provides a method for synthesizing substitutable building block E′ with 4,6-0-benzylidene-a-p-methoxyphenyl-D-glucose (the process is the same as that of building block F), as shown in FIG. 11, and the conditions are as follows: a) Ac₂O, Pyridine, 96%; b) 60% HAc, 60° C., 82%; c) 1. DAIB, TEMPO, ACN/H₂O; 2. BnBr, K₂CO₃, DMF (two-step 76%); d) CH₃COCH₂CH₂COOH, DMAP, DCC, Dioxane (88%); e) 1. CAN, ACN/toluene/H₂O; 2. CNCCl₃, K₂CO₃, CH₂Cl₂ (M-step yield 66%).

Preparation of E1′: dissolve 4,6-0-benzylidene-a-p-methoxyphenyl-D-glucose (37.4 g) in pyridine (200 ml), cool the mixture to 0° C., and add acetic anhydride (100 ml). Stir at this temperature for 1 hour, and naturally rise to room temperature to react overnight. Add methanol for quenching, then distill and concentrate it under reduced pressure. Dissolve the residue ethyl acetate, wash it with 5% sodium hydrogen sulfate aqueous solution, saturated sodium hydrogen carbonate aqueous solution, water and saturated salt solution, dry it with anhydrous sodium sulfate, distill it under reduced pressure, and purify it by silica gel (EA/PE=1/3) to obtain 44 g colorless syrup E1′.

Preparation of E2′: dissolve compound El′ (40 g) in 60% acetic acid solution, and stir at 60° C. for 8 h. After confirmation of complete reaction by TCL method, distill the residue under reduced pressure, and purify it by silica gel (EA/PE=1/3-1/1) to obtain 26.4 g colorless syrup E2′.

Preparation of E3′: dissolve compound E2′ (26.4 g) in acetonitrile/water (300 ml, ACN/H₂0=1/1), add TEMPO (0.7 g) and iodobenzene diacetate (DIAB, 23.5 g) at room temperature, vigorously stir for 3 hours, add methanol for quenching, distill and concentrate the residue under reduced pressure, and without purification, directly use it for the next reaction. Dissolve the said crude product in DMF (200 ml), add benzyl bromide (21.5 ml) and anhydrous potassium carbonate (48 g), and stir it at room temperature to react overnight. After confirmation of complete reaction by TCL method, filter it on diatomite filter plate, distill the filtrate liquor under reduced pressure. Add ethyl acetate to the residue, wash it with water and saturated saline, dry it with anhydrous sodium sulfate, and distill it under reduced pressure. Purify the residue by silica gel (EA/PE=1/1) to obtain 25.8 g white solid E3′. The measured value of ESI-MS M/Z is 474.2, and the theoretical calculated value is 474.15.

Preparation of E4′: dissolve compound E3′ (21 g) in dioxane (200 ml), add levulinic acid (8.2 g), DCC (14.0 g) and DMAP (0.8 g), stir for 2 hours at room temperature, add precooled ether (350 ml), filter, and wash the filtrate liquor repeatedly with sodium bisulfate aqueous solution and water, dry it with anhydrous magnesium sulfate, distill it under reduced pressure, and purify it by silica gel (EA/PE=1/2) to obtain colorless syrup E4′.

Preparation of E5′: dissolve compound E4′ (15 g) in acetonitrile/toluene (200 ml, ACN/Toluene=1.5/1), cool the mixed solution to 0° C., slowly add cerium (IV) ammonium nitrate (14 g dissolved in 100 ml water), stir at this temperature for 30 min, and then add ethyl acetate to dilute the mixed solution. Wash the organic phase with saturated sodium bicarbonate solution and saturated salt water, dry it with anhydrous sodium sulfate and distill it under reduced pressure. Purify it by silica gel (EA/PE=1/2) to obtain colorless syrup. Dissolve the said colorless syrup in dichloromethane (120 ml), then add trichloroacetonitrile (15 ml) and anhydrous potassium carbonate (20 g), stir for 2 hours at room temperature, filter, distill the filtrate liquor under reduced pressure, and purify the residue by silica gel (EA/PE=1/4-1/2) to obtain 10.3 g foamy white solid E5′.

### EXAMPLE 12

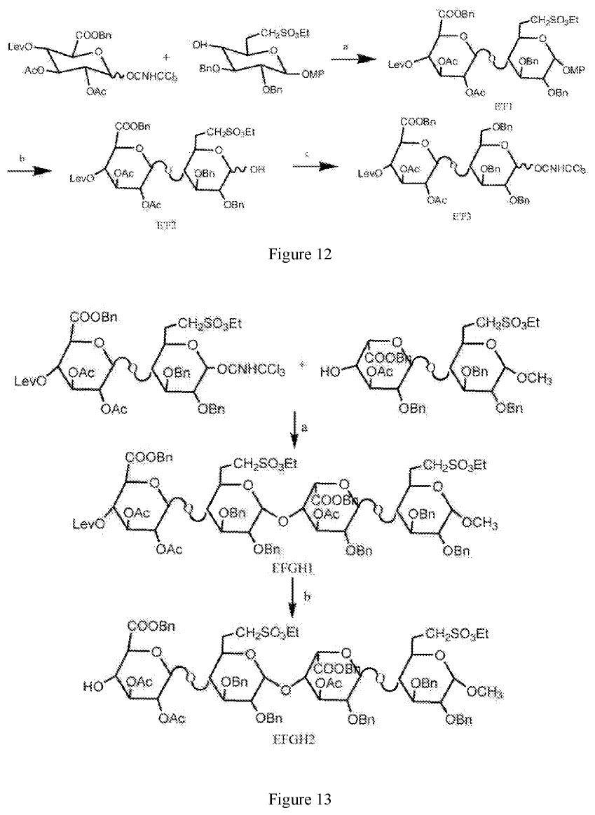

The example provides a method for synthesizing disaccharide E′F with substitutable building block E5′ and building block F4, as shown in FIG. 12, and the conditions are as follows: a) TMSOTf, 4AMS, CH₂Cl₂, 79%; b) CAN, ACN/toluene/U0, 89%; c) CNCCl₃, K₂CO₃, CH₂Cl₂ 74%.

Preparation of E′F1: dissolve substitutable building block E5′ (19.1 g) and F4 (14.3 g) in dry dichloromethane (350 ml), add newly activated 4 A molecular sieve (16 g), stir at room temperature for 30 min, then cool the mixture to −20° C., add TBSOTf solution (1.5 ml, dissolved in 25 ml dichloromethane) dropwise, stir at this temperature for 1 hour, then rise to room temperature, and continue to react for 3 hours. After confirmation of complete reaction by TCL method, neutralize it with triethylamine. Filter the mixed solution with diatomaceous earth, wash the filtrate liquor with water and saturated salt water, dry it with anhydrous sodium sulfate, and distill it under reduced pressure. Purify the residue by silica gel (EA/PE=1/2) to obtain 20.2 g product. The measured value of ESI-MS M/Z is 1022.4, and the theoretical calculated value is 1022.36.

Preparation of E′F2: dissolve E′F1 (10.2 g) in acetonitrile/toluene (200 ml, ACN/Toluene=1.5/1), cool the mixed solution to 0° C., and slowly add cerium (IV) ammonium nitrate (16.4 g dissolved in 100 ml water), stir at this temperature for 30 min, add ethyl acetate into the mixed solution for dilution, wash the organic phase with saturated sodium bicarbonate solution and saturated salt water, dry it with anhydrous sodium sulfate, and distill it under reduced pressure. Purify it by silica gel (EA/PE=1/2) to obtain colorless syrup E′F2 (8.2 g). The measured value of ESI-MS M/Z is 916.3, and the theoretical calculated value is 916.32.

Preparation of E′F3: dissolve E′F2 (8.2 g) in dichloromethane (100 ml), add trichloroacetonitrile (12 ml) and anhydrous potassium carbonate (15 g), stir for 3 hours at room temperature, filter, distill the filtrate liquor under reduced pressure, and purify the residue by silica gel (EA/PE=1/4-1/2) to obtain 6.9 g foamy white solid. The measured value of ESI-MS M/Z is 1043.3, and the theoretical calculated value is 1043.27. ¹H-NMR (600 Hz, CDCl₃) δ7.31-7.47 (m,15H,Ar—H), 5.65 (d, 1H, F-1), 5.34-5.41 (m,2H, C00CH2Ph)5.14(m,1H,E-2), 4.89-4.71 (m,2H,E-3,E-4), 4.45-4.64 (m,5H,ET,Ph-CH2), 4.21 (q,2H,SO3CH2CH3), 3.45-3.84 (m,3H,F-2,F-4,F-5), 3.80(s,0CH3), 3.29-3.15(m,2H, F-3,F-7a), 3.09 (m,1H,F-7b), 2.60-2.72 CH3C0CH2CH2C00), 2.40-2.28 (m,1H,H-6a), 2.10(s,3H,CH3C0CH2CH2C00) 1.95-1.84(m,1H,H-6b), 1.34 (t,3H,SO3CH2CH3).

### EXAMPLE 13

The example provides a method for synthesizing tetrasaccharide EFGH with building block E′F3 and disaccharide building block GH, as shown in FIG. 13, and the conditions are as follows: a) TMSOTf, 4 ÅMS, CH₂Cl₂, 73%; b) NH2NH2, HAc, Pyridine, 82%.

Preparation of tetrasaccharide E′EFGH1: dissolve building block E′F3 (13.1 g) and GH4 (8.8 g) in dry dichloromethane (200 ml), add newly activated 4 A molecular sieve (10 g), stir at room temperature for 30 min under the protection of argon, then cool the mixture to −20° C., add TBSOTf solution (0.5 ml dissolved in 20 ml dichloromethane) dropwise, stir at this temperature for 1 hour, then rise to room temperature, and continue to stir for 3 hours. After confirmation of complete reaction by TCL method, neutralize it with triethylamine. Filter the mixed solution with diatomaceous earth, wash the filtrate liquor with water and saturated salt water, dry it with anhydrous sodium sulfate, and distill it under reduced pressure. Purify the residue by silica gel (EA/PE=1/2-1/1) to obtain 13.0 g foamy solid EFGH1. The measured value of ESI-MS M/Z is 1778.7, and the theoretical calculated value is 1778.65.

Preparation of Tetrasaccharide EFGH2: dissolve tetrasaccharide EFGH1 (10 g) in pyridine (70 ml), add acetic acid (80 ml) and monohydrate hydrazine hydrate (7.5 ml) at room temperature, stir for 15 minutes, then pour the mixed solution into water (300 ml), extract with ethyl acetate, wash the organic phase dilute salt acid (0.2M, 300 ml) and water, purify the residue silica gel (EA/PE=1/3-1/1) to obtain 7.8 g white solid EFGH2. The measured value of ESI-MS M/Z is 1680.6, and the theoretical calculated value is 1680.61. ¹H-NMR (600 MHz, CDCl₃): δ7.18-7.37 (m,35H), 5.56 (d,1H), 5.25 (d,1H), 5.17 (d,1H), 4.83-5.01 (m,12H) , 4.24-4.30 (m,2H), 4.01(t,1H), 3.85 (s,3H), 3.59-3.84(m,6H), 3.29-3.57 (m,6H), 3.09-3.25(m, 6H), 2.19-2.41 (m,2H), 2.01-2.03 (s,9H), 1.78-1.94 (m, 2H), 1.4 (t,6H).

### EXAMPLE 14

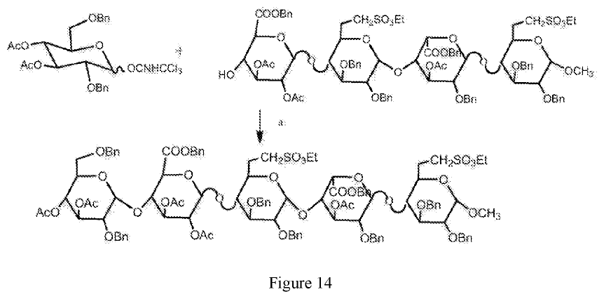

The example provides a method for synthesizing fully-protected pentose DEFGH with building block EFGH and monosaccharide building block D8, as shown in FIG. 14, and under the following conditions: a) TMSOTf, 4 ÅMS, CH₂Cl₂, 72%.

Preparation of pentose DEFGH: dissolve monosaccharide D6 (8.8 g) and tetrose EFGH (16.8 g) in dry dichloromethane (250 ml), add newly activated 4 A molecular sieve (12 g), stir at room temperature for 30 min, then cool the mixture to −20° C., add TBSOTf solution (0.6 ml dissolved in 50 ml dichloromethane) dropwise, stir at this temperature for 1 hour, then rise to room temperature, and continue to stir for 3 hours. After confirmation of complete reaction by TCL method, neutralize it with triethylamine. Filter the mixed solution with diatomaceous earth, wash the filtrate liquor with water and saturated salt water, dry it with anhydrous sodium sulfate, and distill it under reduced pressure. Purify the residue by silica gel (EA/PE=1/3-1/2) to obtain 15.0g foamy solid, the fully-protected pentose DEFGH.

### EXAMPLE 15

The pentose (API) in Example 10 is subjected to a biological test: anticoagulant factor Xa activity and half-life (Ti/2), as shown in Table 1. Determination of anticoagulant factor Xa activity: Refer to substrate developing method for determination of anticoagulant factor Xa of low molecular heparin. Measurement of half-life: inject this anticoagulant pentose (dosage 1 mg/Kg) in male Wistar rats intravenously for pharmacokinetic study, calculate the concentration of this compound in blood by measuring the anticoagulant factor Xa in plasma, and calculate the half-life (T1/2) by the concentration-time curve. The result shows that the pentose in this patent application has good anticoagulant effect.

### COMPARATIVE EXAMPLE 1

This example provides a pentose compound, which is basically the same as the main structure in Example 10, except that the structure of monosaccharide unit F and monosaccharide unit H is different, and the specific structure is as follows:

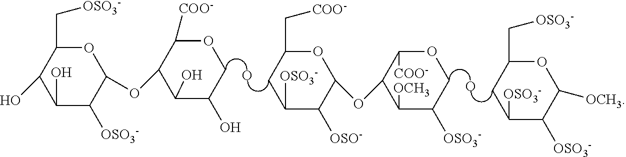

TABLE 1
Biological Test Data Sheet of Pentose in Example 15.

| | | Anticoagulant | |
|---|---|---|---|
| | | Factor Xa | Half-life |
| Example | The Compound | (IU/mg) | (H) |
| Example 15 | API | 1560 = 65 | 0.7 ± 0.1 |
| Available in the | Fondaparinux | 750 + 30 | 0.6 ± 0.1 |
| Market | sodium | | |
| Comparative | | 1270 ± 45 | 0.8 ± 0.1 |
| Example 1 | | | |

The said examples are only for explaining the technical concept and characteristics of the present invention, and their purpose is to enable people familiar with this technology to understand the content of the present invention and implement it accordingly, but not to limit the scope of protection of the present invention. All equivalent changes or modifications made according to the spirit of the present invention shall be covered within the scope of protection of the present invention.

1-13. (canceled)

14. A compound with high anticoagulant activity, wherein the compound is a pentose compound including connected monosaccharide units D, E, F, G and H in sequence through glycosidic bonds, wherein a spatial configuration of bond connections is α-D-glucose-(1→4)-O-β-D-glucuronic acid-(1→4)-O-α-D-(6-Carbon substitution for oxygen)glucose-(1→4)-O-α-L-iduronic acid-(1→4)-O-α-D-(6-Carbon substitution for oxygen)methylglucose; wherein the monosaccharide unit D is a glucose 2,6-O-sulfated group, wherein the monosaccharide unit E is a glucuronidated group, wherein the monosaccharide unit F is a glucose 2,3-O-6-Carbon substitution for oxygen-sulfated group, wherein the monosaccharide unit G is a L-iduronic acid 2-O-sulfated group, and wherein the monosaccharide unit H is a glucose 2,3-O-6-Carbon substitution for oxygen-sulfated group.

15. The compound of claim 1, wherein the compound is an ionic compound having a structure of formula (1):

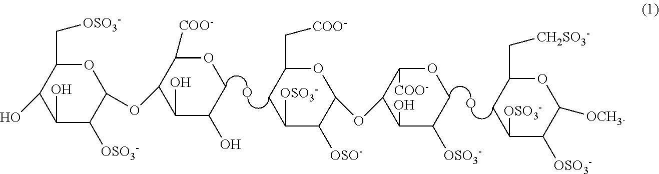

16. The compound of claim 15, wherein the compound has a cation of potassium ion, sodium ion, hydrogen ion, or combinations thereof.

17. The compound of claim 14, wherein the compound is used as an active ingredient for preparing drugs for anticoagulant dysfunction.

18. The compound of claim 14, wherein the compound is mixed with at least one pharmaceutical molding agent in a unit dose of 0.l-10 mg.

19. A method for synthesizing a compound with high anticoagulant activity according to claim 1 characterized in that a trisaccharide composed of monosaccharide units D, E and F is connected with a disaccharide composed of monosaccharide units G and H; or a disaccharide composed of monosaccharide units D and E is connected with a trisaccharide composed of monosaccharide units F, G and H; or a tetrasaccharide composed of monosaccharide units D, E, F and G is connected with a monosaccharide composed of monosaccharide unit H; or a disaccharide composed of monosaccharide units D and E is connected with a disaccharide composed of monosaccharides unit F and G and subsequently connected with a monosaccharide composed of monosaccharide unit H.

20. The method of claim 19, wherein a trisaccharide composed of monosaccharide units D, E, and F are synthesized, wherein the trisaccharide has a structure of formula (2): 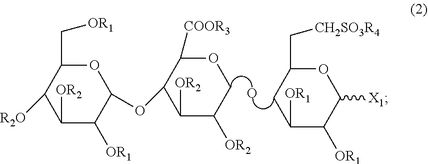
wherein X1 is selected from thioalkyl, thioaryl, halogen, trichloroiminoacetyl, phosphate ester, phosphate ester leaving group, or n-pentenyl group, and wherein the spatial configuration is α or β; R1 is benzyl; R2 is selected from alkyl acyl, aryl acyl, or alkyl aryl acyl; R3 is benzyl; R4 is alkyl;
synthesizing a disaccharide composed of monosaccharide units G and H is synthesized, and its structural general formula is shown in formula (3): 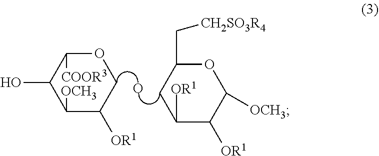
wherein R1 is benzyl; R2 is selected from alkyl acyl, aryl acyl, or alkyl aryl acyl; R3 is benzyl; R4 is alkyl; and
bonding the trisaccharide to the disaccharide.

21. The method of claim 19, wherein a tetrasaccharide composed of monosaccharide units E, F, G, and H is synthesized, and wherein the tetrasaccharide has the structure of formula (4): 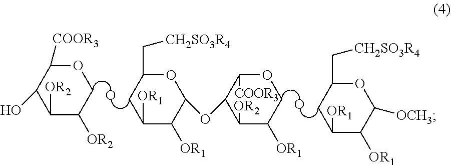
wherein R1 is benzyl; R2 is selected from alkyl acyl, aryl acyl, or alkyl aryl acyl; R3 is benzyl; R4 is alkyl; and
bonding the tetrasaccharide to a monosaccharide of formula (5): 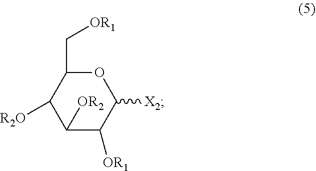
wherein X2 is selected from thioalkyl, thioaryl, halogen, trichloroiminoacetyl, or n-pentenyl group, having a spatial configuration of α or β;
R1 is benzyl;
R2 is selected from alkyl acyl, aryl acyl, or alkyl aryl acyl.

22. The method of claim 19, wherein the monosaccharide unit D is derived from monosaccharides with the following structural general formula: 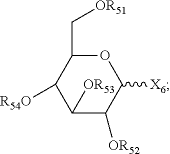
wherein X6 is selected from thioalkyl, thioaryl, halogen, trichloroiminoacetyl, or n-pentenyl, having a spatial configuration of α or β;
R51 is benzyl;
R52 is benzyl;
R53 is selected from alkyl acyl, aryl acyl, alkyl aryl acyl, allyl, allyl ether, or p-Methoxybenzyl (PMB) protective group;
R54 is selected from alkyl acyl, aryl acyl, or alkyl aryl acyl;
R51 and R54 can form cyclic acetals or ketal.

23. The method of claim 19, wherein the monosaccharide unit E is derived from monosaccharides with the following structural general formula: 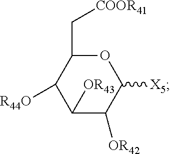
wherein X5 is selected from thioalkyl, thioaryl, halogen, trichloroiminoacetyl, or n-pentenyl, having a spatial configuration of α or β;
R41 is benzyl;
R42 is selected from alkyl acyl, aryl acyl, or alkyl aryl acyl;
R43 is selected from alkyl acyl, aryl acyl, or alkyl aryl acyl;
R44 is selected from hydrogen, chloroacetyl or acetyl propionyl.

24. The method of claim 19, wherein the said monosaccharide unit F is derived from monosaccharides with the following structural general formula: 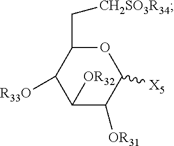
wherein X4 is selected from p-methoxyphenyl or p-methoxybenzyl, having a spatial configuration of α or β;
R31 is benzyl;
R32 is benzyl;
R33 is selected from hydrogen or acetyl propionyl;
R34 is alkyl.

25. The method of claim 19, wherein the said monosaccharide unit G is derived from monosaccharides with the following structural general formula: 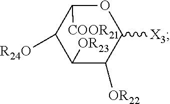
wherein X3 is selected from thioalkyl, thioaryl, trichloroiminoacetyl or n-pentenyl, having a spatial configuration of α or β;
R21 is benzyl;
R22 is selected from benzyl, alkyl acyl, aryl acyl, or alkyl aryl acyl;
R23 is p-methoxybenzyl;
R24 is selected from hydrogen or acetyl propionyl.

26. The method of claim 19, wherein the said monosaccharide unit H is derived from monosaccharides with the following structural general formula: 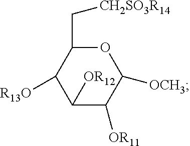
wherein R11 is benzyl;
R12 is benzyl;
R13 is selected from hydrogen or acetyl propionyl;
R14 is alkyl.
